(19) United States
(12) Patent Application Publication
(10) Pub. No.: US 2022/0256837 A1
(43) Pub. Date: Aug. 18, 2022

(54) COMPOSITIONS AND METHODS FOR REDUCING AGGLUTINATION AND IMPROVING HEALTH OF SEX-SORTED SPERM CELLS

(71) Applicant: Inguran, LLC, Navasota, TX (US)

(72) Inventor: Kilby Willenburg, Madison, WI (US)

(73) Assignee: Inguran, LLC, Navasota, TX (US)

(21) Appl. No.: 17/668,910

(22) Filed: Feb. 10, 2022

Related U.S. Application Data
(60) Provisional application No. 63/148,939, filed on Feb. 12, 2021.

(51) Int. Cl. A01N 1/02; C12N 5/076; C12N 5/071; G01N 1/30; G01N 15/14

(52) U.S. Cl. CPC A01N 1/0226; C12N 5/061; C12N 5/0612; G01N 1/30; G01N 2015/1006; G01N 2001/302; C12N 2500/38; C12N 2500/35; C12N 2500/84; G01N 15/1434

(57) ABSTRACT

The invention consists of compositions and methods using low density lipoprotein to reduce agglutination and improve the health of sex-sorted sperm cells.

### REFERENCE TO RELATED APPLICATIONS

This application claims the benefit of U.S. Provisional Patent Application No. 63/148,939 filed Feb. 12, 2021, the entire disclosure of which is incorporated herein by reference.

### BACKGROUND OF THE INVENTION

The livestock industry places a great deal of importance on fertility. Given the prevalence of artificial insemination, and the concomitant reduction in sperm health and fertility associated with the processing sperm cells for use in artificial insemination, there is an ever present need to increase fertility of sperm cells, both conventional (i.e., unsorted sperm cells) and sex-sorted sperm cells.

Sex-sorting sperm cells, or otherwise altering the ratio of viable X chromosome-bearing and Y chromosome-bearing sperm cells in a sample, is known to reduce sperm cell motility, progressive motility, acrosome integrity and viability, among other proxies for sperm health and fertility, as well as actual conception rates. Another known problem associated with sex-sorted sperm cells is the phenomena of agglutination, which results in sperm cells clumping together or adhering to one another. Agglutination impedes the motility of affected sperm cells and may result in a reduction in fertility of the cell sample, as well as possibly signifying cell membrane damage. Accordingly, there is a need to improve health and fertility of sex-sorted sperm cells and, in particular, to reduce the incidence or severity of agglutination.

### SUMMARY OF THE INVENTION

One embodiment of the invention comprises a method of reducing agglutination and improving health of sperm cells having an altered ratio of viable X chromosome-bearing sperm cells to viable Y chromosome-bearing sperm cells comprising staining a sperm cell sample comprising viable X chromosome-bearing sperm cells and viable Y chromosome-bearing sperm cells with a staining media; contacting the stained sperm cell sample with a sheath fluid in a flow path; altering a ratio of the viable X chromosome-bearing sperm cells to the viable Y chromosome-bearing sperm cells to form at least one altered sperm cell population; and collecting the altered sperm cell population in a collection media comprising egg yolk (EY) at a weight/volume (wt./vol.) concentration of 10% or less and a low-density lipoprotein additive, thereby reducing agglutination and improving health of the sperm cells in the collected sperm cell population. In a more specific embodiment, the step of altering a ratio of the viable X chromosome-bearing sperm cells to the viable Y chromosome-bearing sperm cells to form at least one altered sperm cell population comprises i) removing at least a portion of one of either the viable X chromosome-bearing sperm cells or the viable Y chromosome-bearing sperm cells from the sperm cell sample or ii) photo-damaging at least a portion of one of either the viable Y chromosome-bearing sperm cells or the viable X chromosome-bearing sperm cells. In an even more specific embodiment, the method further comprising the steps of injecting the stained sperm cell sample into a flow of sheath fluid; exposing the stained sperm cell sample in the flow of sheath fluid to an electromagnetic radiation source that causes a detectable response in the DNA selective dye; detecting the response of the DNA selective dye to the electromagnetic radiation exposure; analyzing the detected response; and classifying sperm cells in the sperm cell sample based on the analysis of the detected response. In a particular embodiment, the egg yolk is at a wt./vol. concentration between 0.01% to 10%. In an even more particular embodiment, the egg yolk is at a wt./vol. concentration of between 0.01% to 5%. In an even yet more particular embodiment, the egg yolk is at a wt./vol. concentration of between 0.01% to 1%. In a yet even more particular embodiment, the egg yolk is at a wt./vol. concentration of between 0.01% to 0.5%. In yet another embodiment, the egg yolk is at a wt./vol. concentration of between 0.01% to 0.05%. In a further embodiment, the egg yolk is at a wt./vol. concentration of 0.01%. In another embodiment of the invention, the egg yolk is at a wt./vol. concentration of 0%. In a particular embodiment of the invention, the low-density lipoprotein additive is extracted from pasteurized and/or liquid egg yolk. In another embodiment, the low-density lipoprotein additive is at a wt./vol. concentration of between 0.5% to 30%. In yet another embodiment, the low-density lipoprotein additive is at a wt./vol. concentration of between 0.5% to 10%. In an even further embodiment, the low-density lipoprotein additive is at a wt./vol. concentration of between 0.5% to 5%. In another embodiment, the low-density lipoprotein additive is at a wt./vol. concentration of between 1% to 5%. In a particular embodiment of the invention, the low-density lipoprotein additive is at a wt./vol. concentration of 2.5%. In an even more particular embodiment, the low-density lipoprotein additive is at a wt./vol. concentration of between 1% to 5% and the egg yolk is at a wt./vol. concentration of between 0.01% to 1%. In a specific embodiment, the sperm cells are porcine sperm cells or bovine sperm cells. In another embodiment, the step of collecting is conducted at a temperature less than 37° C. In a further embodiment, the step of collecting is conducted at a temperature less than 22° C. In a further embodiment, the step of collecting is conducted at a temperature less than 10° C. In a particular embodiment of the invention, the low-density lipoprotein additive is extracted using polyethylene glycol (PEG). In an even more particular embodiment, the collection media further comprises vitamin B or alpha-ketoglutarate.

The invention also encompasses a composition comprising egg yolk at a wt./vol. concentration of between 0.01% to 0.05%; a low-density lipoprotein additive at a wt./vol. concentration of between 1% to 5%; and a sperm cell sample comprising an altered ratio of viable X chromosome-bearing sperm cells to viable Y chromosome-bearing sperm cells. In a particular embodiment, the low-density lipoprotein additive is extracted from pasteurized and/or liquid egg yolk.

In another embodiment, the sperm cell sample in the composition has an agglutination rank of 2 or less.

### BRIEF DESCRIPTION OF THE DRAWINGS

FIG. 1 is a diagram illustrating sex-sorting sperm with a flow cytometer.

FIG. 2 is a diagram illustrating a microfluidic chip that may be used to sex-sort sperm.

### DETAILED DESCRIPTTION OF THE INVENTION

The invention broadly encompasses compositions and methods for reducing agglutination and improving the health of sperm cells that have been subjected to a process for altering the ratio of viable X chromosome-bearing sperm cells to viable Y chromosome-bearing sperm cells, such as flow cytometric sex-sorting. Specifically, by collecting such sperm cells in a collection media comprising a relatively low concentration of egg yolk and a low-density lipoprotein additive, agglutination is significantly reduced and the health and viability of the sperm cells is significantly improved.

### Altering the Ratio of X Chromosome-Bearing and Y Chromosome-Bearing Sperm Cells

Commonly used and well known methods for altering the ratio of X chromosome-bearing and Y chromosome-bearing sperm cells involve the use of flow cytometry systems, as exemplified by and described in U.S. Pat. Nos. 5,135,759, 5,985,216, 6,071,689, 6,149,867, and 6,263,745; International Patent Publications WO 99/33956 and WO 01/37655; and U.S. patent application Ser. No. 10/812,351 (corresponding International Patent Publication WO 2004/088283), the content of each of which is hereby incorporated herein by reference. Altering the ratio of X chromosome-bearing and Y chromosome-bearing sperm cells may be accomplished using any process or device known in the art for cell analysis, sorting and/or population enrichment, including but not limited to use of a flow cytometer (which includes devices such as a microfluidic chip), and encompasses techniques for physically separating X and Y bearing sperm from each other, as with droplet sorting and fluid switching sorting, and as well as techniques in which sperm bearing the undesired sex chromosome are killed, immobilized, or otherwise rendered infertile, such as by use of laser ablation/photo-damage techniques. Based on the fluorescence emitted by a DNA selective dye upon exposure to a light source such as a high intensity laser beam, a flow cytometer (including a microfluidic device) is able to measure or quantify the amount of DNA present in each cell stained with the DNA selective dye. Because the DNA content of X chromosome-bearing sperm cells and Y chromosome-bearing sperm cells is different in many species, X chromosome-bearing sperm cells and Y chromosome-bearing sperm cells stained with a DNA-selective dye are able to be differentiated by flow cytometric analysis.

Generally, a sperm cell sample to by analyzed via a flow cytometer (including a microfluidic device) is contained in a sample fluid. A sheath fluid is generally used in a flow cytometer or microfluidic device to hydrodynamically focus, entrain or orient sperm or nuclei in the sample fluid. Generally, the sheath fluid is introduced into a nozzle of a flow cytometer or into a microfluidic device using pressurized gas or by a syringe pump. The pressurized gas is often high quality compressed air. In certain embodiments of the invention, a stream containing sperm cells to be analyzed may be comprised of a sample fluid and a sheath fluid, or a sample fluid alone. Optionally, the sample fluid or sheath fluid may also contain an additive, such as, one or more antioxidants, an antibiotic or a growth factor. Each of these additives may be added to either fluid.

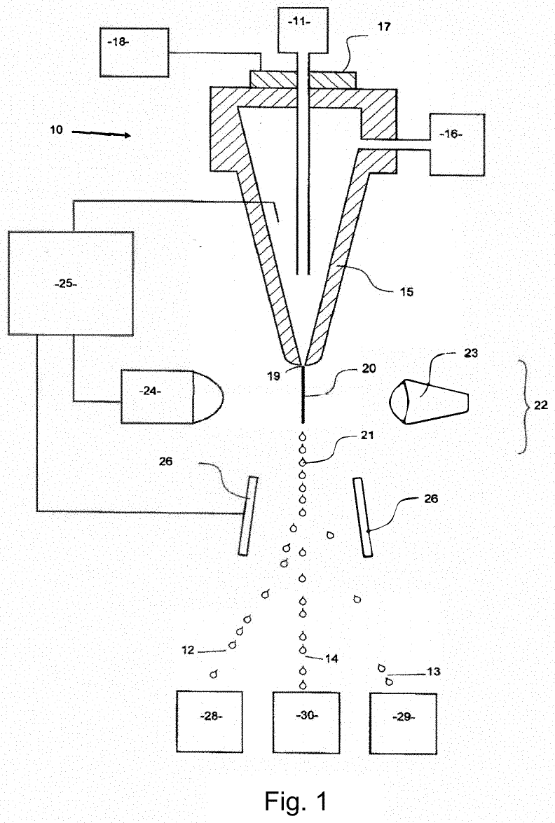

FIG. 1 illustrates, in schematic form, one embodiment of part of a flow cytometer used to analyze and then sort a sperm cell sample to form one or more subpopulations, the flow cytometer being generally referenced as 10. The flow cytometer 10 of FIG. 1 can be programmed by an operator to generate two charged droplet streams, one containing X chromosome-bearing cells charged positively 12, for example, one containing Y chromosome-bearing cells charged negatively 13 for example, while an uncharged undeflected stream of indeterminate or undesired sperm cells simply goes to waste, each stream collected in receptacles 28, 29 and 30, respectively.

Initially, a stream of sperm cells under pressure, is deposited into the nozzle 15 from the sperm cell source 11 in a manner such that they are able to be coaxially surrounded by a sheath fluid supplied to the nozzle 15 under pressure from a sheath fluid source 16. An oscillator 17 which may be present can be very precisely controlled via an oscillator control mechanism 18, creating pressure waves within the nozzle 15 which are transmitted to the coaxially surrounded sperm cell stream as it leaves the nozzle orifice 19. As a result, the exiting coaxially surrounded sperm cell stream 20 could eventually and regularly form droplets 21.

The charging of the respective droplet streams is made possible by the cell sensing system 22 which includes a laser 23 which illuminates the nozzle exiting stream 20, and the light emission of the fluorescing stream is detected by a sensor 24. The information received by the sensor 24 is fed to a sorter discrimination system 25 which very rapidly makes the decision as to whether to charge a forming droplet and if so which charge to provide the forming drop and then charges the droplet 21 accordingly.

A characteristic of X chromosome-bearing sperm cells is that they absorb more fluorochrome dye than Y chromosome-bearing sperm cells or nuclei because of the presence of more DNA, and as such, the amount of light emitted by the laser excited absorbed dye in the X chromosome-bearing sperm cells differs from that of the Y chromosome-bearing sperm cells. One of the difficulties in accurate quantification of sperm DNA using fluorescence is the geometry of the sperm head, which is shaped like a paddle in most species. Generally, the intensity of fluorescence is lowest when the flat face of the sperm is oriented toward a fluorescence detector. This flat orientation actually results in the most accurate measure of DNA content within a cell and thus, in sex sorting applications, the best discrimination between X and Y chromosome-bearing sperm subpopulations. There are many techniques known in the art used to orient sperm using various forces generated by the flow cytometer and/or microfluidic device, all of which are contemplated for use with the invention. One way in which orientation can be accomplished in a flow cytometer is by using an orienting nozzle such as described in U.S. Pat. No. 6,357,307, which is hereby incorporated by reference in its entirety. In one embodiment of the invention, two detectors are used for detecting fluorescence emitted by sperm cells. One of the detectors is oriented at 0° relative to the laser beam or other source of electromagnetic radiation and is used to measure forward fluorescence, which corresponds to cell DNA content. The second detector is oriented 90° relative to the laser beam and is used to measure side fluorescence, which corresponds to the orientation of the sperm cell. Since the fluorescence signal is highest for sperm cells oriented with their paddle edge toward the side fluorescence detector, only the sperm cells that emit peak fluorescence to the side fluorescence detector are considered oriented by the flow cytometer.

The charged or uncharged droplet streams pass between a pair of electrostatically charged plates 26, which cause them to be deflected either one way or the other or not at all depending on their charge into respective collection vessels 28 and 29 to form a subpopulation of X-chromosome-bearing sperm cells and a subpopulation of Y chromosome-bearing sperm cells, respectively. The uncharged non-deflected sub-population stream containing undesired or indeterminate cells go to the waste container 30.

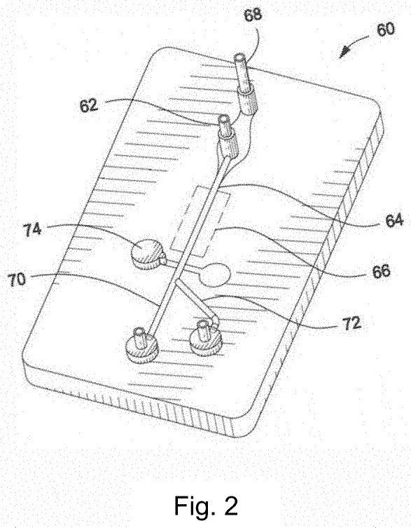

Turning now to FIG. 2, an alternative sperm cell sorting instrument or flow cytometer is partially illustrated in the form of a microfluidic chip 60. The microfluidic chip 60 may include a sample inlet 62 for introducing sample containing cells into a fluid chamber 64 and through an inspection zone 66. Sample introduced through the sample inlet 62 may be insulated from interior channel walls and/or hydrodynamically focused with a sheath fluid introduced through a sheath inlet 68. Sample may be interrogated at the inspection zone 66 with an electromagnetic radiation source (not shown), such as a laser, arc lamp, or other source of electromagnetic electricity. Resulting emitted or reflected light may be detected by a sensor (not shown) and analyzed with an analyzer (not shown). Each of the sheath pressure, sample pressure, sheath flow rate, and sample flow rate in the microfluidic chip may be manipulated in a manner similar to a jet-in-air flow cytometer, by either automatic adjustments performed by the execution of written instructions in the analyzer or by manual adjustments performed by an operator.

In certain embodiments of the invention, once inspected, sperm cells in the fluid chamber 64 may be mechanically diverted from a first flow path 70 to a second flow path 72 with a separator 74, for altering fluid pressure or diverting fluid flow. The sperm cells may also be permitted to continue flowing along the first flow path (70) for collection. The illustrated separator (74) comprises a membrane which, when depressed, may divert particles into the second flow path (72). Other mechanical or electro-mechanical switching devices such as transducers and switches may also be used to divert sperm cell flow.

Collection of Processed Sperm Cells into Collection Media

Once the ratio of X chromosome-bearing sperm cells and Y chromosome-bearing sperm cells in a sample has been altered by the flow cytometer, the sperm cells are collected in a vessel that contains a collection, or “catch,” media. Generally, the purpose of the collection media includes providing a fluid support for the cells and is comprised of a low-density lipoprotein additive at a wt./vol. concentration of, between 0.5% to 30%, between 0.5% to 10%, between 0.5% to 5%, between 1% to 5%, 0.5%, 1%, 2%, 2.5%, 3%, 4% or 5%.

In a further aspect of the invention, the collection media is further comprised of egg yolk at a wt./vol. concentration of, less than 30%, less than 20%, less than 10%, less than 5%, less than 1%, less than 0.5%, less than 0.05%, 0.01%, between 0.01% to 10%., between 0.01% to 5%, between 0.01% to 1%, between 0.01% to 0.5%, between 0.01% to 0.05%, or 0%.

Additionally, protein sources other than egg yolk and low-density lipoprotein may be included in a collection media of the invention, including but not limited to lecithin, casein and albumin.

In some embodiments of the invention, a collection media of the invention may comprise a suitable buffer, including but not limited to, HEPES ((4-(2-hydroxyethyl)-1-piperazineethanesulfonic acid)), sodium bicarbonate, MOPS ((3-(N-morpholino)propanesulfonic acid)), TRIS (tris(hydroxymethyl)aminomethane), TRIS-citrate, TES (2-[[1,3-dihydroxy-2-(hydroxymethyl)propan-2-yl]amino]ethanesulfonic acid), TALP (Tyrode's Albumen Lactate Pyruvate), TCA (trichloroacetic acid), PBS (phosphate buffered saline), milk, derivatives thereof and combinations thereof.

In certain embodiments of the invention, a collection media may comprise a suitable energy source, such as a monosaccharide such as fructose, glucose, or mannose, or even a disaccharide or trisaccharide.

The collection fluid may also comprise a suitable sugar alcohol, including ethylene glycol, glycerol, erythritol, threitol, arabitol, ribitol, xylitol, sorbitol, galactitol, iditol, volemitol, fucitol, inositol, a glycylglycitol, or combinations thereof or a suitable glycol, such as propylene glycol, butane triol or combinations thereof. In certain embodiments, the collection media may include an amount of such a sugar alcohol between at a concentration by wt./vol. or vol./vol. of between about 1% and about 2%; between about 2% and about 4%; between about 4% and about 6%; between about 6% and about 8%; between about 3% and about 7%.; or between about 3.5% and about 5.5%.

Optionally, the collection media may also contain additives such as, an antibiotic, a growth factor or one or more antioxidants or scavengers of reactive oxygen species. Suitable antioxidants or scavengers of reactive oxygen species include, but are not limited to, catalase, SOD, an SOD mimic, glutathione, glutathione reductase, glutathione peroxidase, pyruvate, caproic acid, mercaptoethanol, BHT, lipoic acid, flavins, quinines, vitamin K (and related vitamers), vitamin B12, vitamin B12 vitamers, vitamin E (and related vitamers), tocopherols, tocotrienols, α-tocopheryl, alpha ketoglutarate (AKG), malondialdehyde (MDA), asymmetric dimethylarginine (ADMA) and biologically active derivatives thereof, and combinations thereof.

Collection media may also be kept at various temperatures before, during and after collecting sperm cells subjected to a process that alters the ratio of sex chromosomes in a sperm cell sample. Collection media may be at temperatures at or below room temperature (22° C.) as well as a temperature, between 10 to 20° C., between 5 to 10° C., between 0 to 5° C., of 5° C., or of 0° C., before, during and after sperm cell collection.

### Extraction of LDL

Any method for extracting LDL from egg yolk that in known in the art may be used in the invention. By way of example only, the following method for extracting LDL (using polyethylene glycol or “PEG”) may be used in the invention:

Preparation of 0.17 M NaCl Solution

1. Combine 100 mL of Milli-Q water with 0.993 g NaCl in a sterile 250 mL bottle.

2. Place solution on stir plate until dissolved.

3. Place solution in autoclave for sterilization.

Preparation of 50% PEG Solution

1. Place 100 mL Milli-Q water in a sterile 250 ml media bottle.

2. Weigh 100 g of PEG 4000.

3. Place bottle on stir plate and add PEG in ˜20 g portions.

4. Stir for 1 hr at room temperature.

Egg Yolk Plasma Extraction

1. Dilute 100 mL of liquid egg yolk with 100 mL of 0.17 M NaCl solution in sterile 500 mL bottle and stir for 1 hr at 4° C.

2. Transfer EY-NaCl solution to ultracentrifuge tubes and spin at 18,500×g for 15 min at 4° C.

3. Aspirate the top white lipid layer from the tubes.

4. Remove supernatant (i.e., yolk plasma) and transfer to another a clean ultracentrifuge tube.

5. Centrifuge plasma at 15,000×g for 15 min at 4° C.

6. Remove supernatant and transfer to a 250 mL beaker.

PEG Precipitation (10% PEG Added to Egg Plasma)

1. Add PEG solution at 10% of total weight of egg plasma
a. Ex: 100 g egg plasma+10 g PEG solution

2. Place sterile beaker on scale and weigh desired amount of egg plasma.

3. Add correct volume of PEG.

4. Place egg plasma/PEG solution on stir plate and cover with aluminum foil.

5. Stir for 1 hr at room temperature.

6. Transfer solution to ultracentrifuge tubes and spin at 18,500×g for 20 min at 4° C.

7. Prepare for removal of supernatant from each centrifuge tube (See Picture1).

8. Place sterile 500 mL bottle on scale and tare.

9. Remove a small portion of the supernatant to aspirate or decant the liquid portion.

10. Scrape off and discard the white layer on the supernatant that was touching the liquid and pellet.

11. Remove remaining supernatant by scraping LDL from the tubes with a spatula and place in bottle.

12. If not making media, place LDL's in 50 mL conical tubes, gas with nitrogen and store at −20° C.

In certain embodiments of the invention, LDL is extracted from pasteurized and/or liquid egg yolk.

### EXAMPLES

### Introduction:

Cells that are flow sorted are typically collected in a conical tube containing catch fluid, which is an egg yolk-based media. Finally, cells are concentrated by centrifugation and resuspended with a long-term media intended for long term preservation.

The following examples are based on catch media with focus on replacing the egg yolk with low density lipoprotein (LDL) harvested from egg yolk (EY). LDL's are the principal component in egg yolk, representing ⅔ of the dry matter. They are spherical particles composed of a nucleus of lipids (triglycerides and cholesterol) surrounded by phospholipids and proteins, which play a role in the stabilization of the LDL structure. Collectively, they consist of 83-89% lipid and 11-17% protein with a pH between 6.3 and 7.5. The use of LDL's was tested as an EY replacement in boar sperm catch media.

The following Examples summarize the use of LDL, EY, various EY sources for LDL preparation and a combination of EY and LDL in boar catch media.

### Statement of the Problem(s):

Despite significant improvement in post sort survivability, conventional (i.e. non sorted) sperm have higher motility and viability compared to sorted sperm after 72 hrs. The addition of LDL as a replacement for the protein source in EY could offset these disparities between sorted and non-sorted sperm in fresh, aged semen.

It has been shown that sorted sperm tend to agglutinate, which could be from the sorting process itself, effect of EY or a combination of the two. Therefore, the addition of LDL could minimize agglutination.

### LDL Preparation:

Initial experiments with LDL's were prepared by dialysis in a method described by Moussa et al., 2002. However, this method is very labor intense with inconsistencies in the purity and level of extraction, so all data after Example 1 was from LDL's prepared by ultracentrifugation in a modified protocol reported by Wang et al., 2018.

The concentration of LDL's in boar catch media was unknown in these first experiments so we tested the following:

Compare 20% EY vs 20% LDL in catch media.

Find the optimal level of LDL's in boar catch media.

Find the optimal level of EY in boar catch media.

### Example 1: The Addition of LDL to Boar Catch Media on Post Sort Motility and Viability

Materials and Methods: Boars (n=4) over 3 replicates were used in the experiment. Boars were collected and sorted according to the boar sorting protocol. Motility and viability were analyzed every 24 hrs for 72 hrs.

### Treatments:

Control: 20% EY catch media

20% LDL: 20% LDL Catch media

TABLE 1
LsMeans for the comparison of 20% EY catch media (Control)
and 20% LDL catch media on post sort boar sperm total
and progressive (ProgMotil) motility.

| Trt | Motility* | ProgMotil |
|---|---|---|
| Control | 84.05^{A} | 62.16^{A} |
| 20% LDL | 88.9^{B} | 68.5^{B} |
| PSE | 0.43 | 0.79 |

*Superscripts within column are statistically different (P < 0.0001).
PSE = Pooled Standard Errors

TABLE 2
LsMeans for the Comparison of 20% EY catch media (Control)
and 20% LDL catch media on post sort boar sperm viability.

Viability*

| Trt | 0 h | 24 h | 48 h | 72 h |
|---|---|---|---|---|
| Control | 88.31 | 79.48^{A} | 74.82^{A} | 74.93^{A} |
| 20% LDL | 91.28 | 88.01^{B} | 88.77^{B} | 88.5^{B} |
| PSE | 1.04 | | | |

*Superscripts within column are statistically different (P < 0.0001).

Summary: Example 1 showed that LDL's appear to improve post sort motility and viability. The next Examples tested different levels of LDL's in boar catch media.

### Example 2: The Effect of LDL Concentration in Boar Catch Media on Post Sort Survivability

Materials and Methods: Boars (n=5) were used in the experiment. Motility, viability, acrosome integrity were analyzed every 24 hrs for 96 hrs.

### Treatments:

+Control: 20% EY catch media
−Control: Non-sorted sperm
0.5: 0.5% LDL in catch media
1: 1% LDL in catch media
5: 5% LDL in catch media
10: 10% LDL in catch media
30: 30% LDL in catch media

TABLE 3
LsMeans for the effect of LDL concentration (0.5, 1, 5, 10, 30%)
in boar catch media compared to a 20% EY and non-sorted control on post
sort motility, viability and PNA⁺ (acrosome damage) sperm.

| Trt | Motility | Viable | PNA+ |
|---|---|---|---|
| Non-sorted | 84.00^{A} | 88.29^{B} | 7.44^{A} |
| 20% EY | 89.63^{B} | 85.00^{C} | 6.00^{B} |
| 0.5 | 89.32^{B} | 86.00^{A} | 2.91^{C} |
| 1 | 90.87^{B} | 88.06^{AB} | 2.45^{C} |
| 5 | 90.48^{B} | 88.13^{AB} | 2.43^{C} |
| 10 | 90.81^{B} | 88.53^{B} | 2.29^{C} |
| 30 | 90.33^{B} | 87.15^{AB} | 2.23^{C} |
| PSE | 1.18 | 0.75 | 0.36 |
| P Value | 0.0006 | 0.0062 | <0.0001 |

Summary: These data reveal that a very low level of LDL's are needed to produce results similar to non-sorted and EY controls. We repeated versions of this experiment several times with all data showing that 1-5% LDL concentrations were the lowest levels to maintain sperm longevity. Unlike, the EY control we did not see any agglutination throughout the LDL treatments. The next Examples focused on finding the minimal level of EY needed to a) reduce agglutination and b) optimize post sort survivability.

### Example 3: The Effect of EY Concentration in Boar Catch Media on Post Sort Survivability

Materials and Methods: Boars (n=5) over 2 replicates were used in the experiment. Motility, viability, acrosome integrity and agglutination were analyzed every 24 hrs for 144 hrs.

### Treatments:

Control: 20% EY catch media
10: 10% EY in catch media
5: 5% EY in catch media
1: 1% EY in catch media
0: 0% EY in catch media

TABLE 4
LsMeans for the effect of EY concentration in
boar catch media on post sort sperm motility.

| Trt | 24 h | 48 h | 120 h | 144 h |
|---|---|---|---|---|
| Control | 87.58 | 85.41 | 82.53^{B} | 79.26^{B} |
| 10 | 84.14 | 82.02 | 79.27^{AB} | 78.29^{B} |
| 5 | 82.41 | 83.42 | 80.62^{AB} | 75.94^{AB} |
| 1 | 83.32 | 81.55 | 75.03^{A} | 69.32^{A} |
| 0 | 43.11^{A} | 36.99^{A} | 20.18 | 19.58 |
| PSE | 2.55 | | | |
| P Value | <0.0001 | | | |

TABLE 5
LsMeans for the effect of EY concentration in
boar catch media on post sort sperm viability.

| Trt | 24 h | 48 h | 120 h | 144 h |
|---|---|---|---|---|
| Control | 85.94 | 81.76^{B} | 77.73^{A} | 74.25^{BC} |
| 10 | 84.82 | 80.55^{AB} | 77.59^{B} | 75.50^{BC} |
| 5 | 83.33 | 78.74^{AB} | 72.87^{A} | 71.45^{AC} |
| 1 | 81.92 | 76.53^{A} | 71.46^{A} | 69.22^{A} |
| 0 | 47.51^{A} | 38.77 | 27.86 | 28.25 |
| PSE | 1.69 | | | |
| P Value | <0.0001 | | | |
| Trt*time | 0.07 | | | |

TABLE 6
LsMeans for the effect of EY concentration in boar
catch media on post sort PNA⁺ sperm (acrosome
damage) and agglutination (Agglutination rank).

| Trt | PNA+ | Agglutination Rank* |
|---|---|---|
| Control | 3.33^{B} | 3.47^{D} |
| 10 | 3.54^{AB} | 2.93^{C} |
| 5 | 4.07^{A} | 2.20^{B} |
| 1 | 4.12^{A} | 1.40^{A} |
| 0 | 12.48^{C} | 1.00^{A} |
| PSE | 0.23 | 0.14 |
| P Value | <0.0001 | <0.0001 |

*Agglutination rank: Subjective rank (1-5) based on the percentage of agglutination within treatment relative to each boar.

Summary: Boar sperm survivability decreased when EY levels fell below 10% in catch media. There were no differences between the standard 20% and 10% concentrations (P>0.1). The higher levels of EY also had more agglutination compared to lower levels of EY, which appeared to affect the accuracy of the CASA to analyze sperm movement. A protein source is needed as shown by the 0% trt, but based on these data we found a reference point for the level of EY for minimal agglutination. Thus, we decided to combine EY and LDL with the intent of minimizing agglutination and maximizing sperm longevity.

### Example 4: LDL/EY Combinations in Boar Catch Media on Post Sort Survivability

### A. Treatments:

−Control: 20% EY catch media
+Control: 0% EY+0.5% LDL catch media
1: 1% EY+0.5% LDL catch media
5: 5% EY+0.5% LDL catch media
N=5 boars
Results: EY control was significantly higher in motility (P<0.0001) and viability (P<0.05) compared to the other treatments. Based on these results we increased the level of LDL in catch media.

### B. Treatments:

−Control: 20% EY catch media
+Control: 0% EY+1% LDL catch media
1: 1% EY+1% LDL catch media
5: 5% EY+1% EY catch media
N=5 boars
Results: There were no differences in motility, viability or acrosome damage (P>0.05) yet, the EY control was numerically higher. However, the percentage of agglutinated cells was significantly higher (P<0.0001) in the EY control compared to the other treatments. From this, the LDL level was increased to 2.5%.

### C. Treatments:

−Control: 20% EY catch media
0.1: 2.5% LDL+0.1% EY catch media
0.5: 2.5% LDL+0.5% EY catch media
1: 2.5% LDL+1% EY catch media

### Results

TABLE 7
LsMeans for the effect of LDL/EY combinations in boar (n = 6) catch
media on post sort motility (total and Progressive (PMot), Viability, Dead,
PNA⁺ sperm (acrosome damage) and agglutination (Agglutination rank (AR)).

| Trt | % EY | % LDL | Motility | PMot | Viability | PNA+ | Dead | AR |
|---|---|---|---|---|---|---|---|---|
| Control | 20 | 0 | 79.24^{A} | 43.13 | 76.52^{A} | 5.63 | 17.88^{A} | 3.35^{B} |
| 0.1 | 0.1 | 2.5 | 77.12^{AC} | 42.47 | 81.82^{B} | 4.91 | 13.6^{B} | 1.05^{A} |
| 0.5 | 0.5 | 2.5 | 76.63^{BC} | 40.42 | 78.56^{AB} | 6.11 | 15.88^{AB} | 1.45^{C} |
| 1 | 1 | 2.5 | 73.36^{B} | 40.89 | 77.5^{A} | 5.84 | 16.55^{A} | 1.6^{C} |
| PSE | | | 1.26 | 1.17 | 1.33 | 0.43 | 1 | 0.14 |
| P Value | | | 0.01 | 0.31 | 0.037 | 0.24 | 0.03 | <0.0001 |

Summary: There were no differences in motile cells in the control treatment compared to 0.1% trt, but there was an improvement in viability favoring the 0.1 treatment compared to the control. There was also a lower % dead and less agglutination for 0.1 treatment as well. These data show that an even lower EY level might be optimal for post sort sperm survival and less agglutination, which prompted the next test.

### D. Treatments:

−Control: 20% EY catch media
+Control: 2.5% LDL catch media
0.05: 2.5% LDL+0.05% EY catch media
0.01: 2.5% LDL+0.01% EY catch media

### Results

TABLE 8
LsMeans for the effect of LDL/EY combinations in boar (n = 6) catch
media on post sort % motility (total and Progressive (PMot), Viability, Dead,
PNA⁺ sperm (acrosome damage) and agglutination (Agglutination rank (AR)).

| Trt | % EY | % LDL | % Motility | % PMot | AR | % Viability | % Dead | % PNA+ |
|---|---|---|---|---|---|---|---|---|
| −Control | 20 | 0 | 79.95 | 41.52^{A} | 3.16 | 73.95^{A} | 19.88^{A} | 6.98^{A} |
| 0.01 | 0.01 | 2.5 | 79.43 | 49.79^{B} | 1.16 | 80.04^{B} | 15.06^{B} | 5.12^{B} |
| 0.05 | 0.05 | 2.5 | 76.94 | 48.7^{B} | 1.44 | 78.1^{B} | 16.12^{B} | 6.07^{AB} |
| +Control | 0 | 2.5 | 77.95 | 48.93^{B} | 1.52 | 79.18^{B} | 15.48^{B} | 5.59^{B} |
| PSE | | | 1.51 | 2.05 | 0.08 | 1.22 | 0.96 | 0.46 |
| P Value | | | 0.48 | 0.01 | <0.0001 | 0.0043 | 0.0025 | 0.039 |

Summary: We see a similar trend with less agglutination in the LDL treatments. There were no differences in total motility, but we did see an improvement in viability, progressive motility and lower % dead in the LDL treatments compared to the EY control (P<0.01). The next experiment was a repeat of the 0.01% EY treatment using additional boars.

### E. Treatments:

−Control: 20% EY catch media
+Control: 2.5% LDL catch media
0.01: 2.5% LDL+0.01% EY catch media

### Results:

TABLE 9
LsMeans for the effect of LDL/EY combinations in boar (n =
9) catch media on post sort motility (total and Progressive
(PMot) and agglutination (Agglutination rank (AR)).

| Trt | % EY | % LDL | % Motility | % PMot | AR |
|---|---|---|---|---|---|
| −Control | 20 | 0 | 79.54^{A} | 47.75^{A} | 2.67^{A} |
| +Control | 0 | 2.5 | 81.51^{AB} | 53.11^{B} | 1.03^{B} |
| 0.01 | 0.01 | 2.5 | 82.13^{B} | 54.05^{B} | 1.08^{B} |
| PSE | | | 0.72 | 1.29 | 0.05 |
| P Value | | | 0.03 | 0.0015 | <0.0001 |

TABLE 10
LsMeans for the effect of LDL/EY combinations in
boar catch media on post sort percent viability.

| | | | Time (h) | | | | |
|---|---|---|---|---|---|---|---|
| Trt | % EY | % LDL | 0 h | 24 h | 96 h | 120 h | 144 h |
| −Control | 20 | 0 | 92.93 | 83.26 | 66.67^{A} | 66.46^{A} | 69.16^{A} |
| +Control | 0 | 2.5 | 92.06 | 83.7 | 83.12^{B} | 85.98^{B} | 80.41^{B} |
| 0.01 | 0.01 | 2.5 | 91.52 | 83.75 | 86.32^{B} | 88.34 | 82.05^{B} |
| PSE | | | 2.02 | | | | |
| P Value | | | <0.0001 | | | | |

TABLE 11
LsMeans for the effect of LDL/EY combinations in
boar catch media on post sort percent PNA⁺ cells.

| | | | Time (h) | | | | |
|---|---|---|---|---|---|---|---|
| Trt | % EY | % LDL | 0 h | 24 h | 96 h | 120 h | 144 h |
| Control | 20 | 0 | 2.26 | 4.69 | 15.17^{A} | 8.58^{A} | 9.36^{A} |
| 2.5 | 0 | 2.5 | 2.45 | 3.77 | 6.14^{B} | 4.86^{B} | 5.65^{B} |
| 0.01 | 0.01 | 2.5 | 2.62 | 3.71 | 4.71^{B} | 3.37^{B} | 4.99^{B} |
| SE | | | 0.93 | | | | |
| P Value | | | <0.0001 | | | | |

TABLE 12
LsMeans for the effect of LDL/EY combinations in
boar catch media on post sort percent dead cells.

| | | | Time (h) | | | | |
|---|---|---|---|---|---|---|---|
| Trt | % EY | % LDL | 0 h | 24 h | 96 h | 120 h | 144 h |
| Control | 20 | 0 | 5.06 | 12.38 | 19.22^{A} | 25.86^{A} | 22.32^{A} |
| 2.5 | 0 | 2.5 | 5.58 | 12.83 | 11.10^{B} | 9.39^{B} | 14.31^{B} |
| 0.01 | 0.01 | 2.5 | 5.98 | 12.71 | 9.34^{B} | 8.54^{B} | 13.31^{B} |
| SE | | | 1.45 | | | | |
| P Value | | | <0.0001 | | | | |

Summary: Numerically, the improvement in total motility is statistically different (P=0.03). Progressive motility was also statistically higher with the 0.01% treatment as well as having a less agglutination. The latter should not be dismissed on the importance of less agglutination. The data for % viability, dead and PNA⁺ are more representative of sperm survivability than the values for motility. A drastic reduction in viable cells was seen around 96 hrs for the control treatments compared to the LDL and LDL/EY treatments, which is not remarkable considering the 9 boars included both “good” and “bad” sorters, so it appears the EY/LDL combo mitigated the effects of sorting for those poor sorting boars. We also saw a similar trend in % dead and % of cells with damaged acrosomes. That said, the combination of 2.5% LDL from egg yolk with 0.01% EY in catch media appeared to optimize post sorting motility and viability with a decrease in cell to cell agglutination.

What we claim is:

1. A method of reducing agglutination and improving health of sperm cells having an altered ratio of viable X chromosome-bearing sperm cells to viable Y chromosome-bearing sperm cells comprising:
staining a sperm cell sample comprising viable X chromosome-bearing sperm cells and viable Y chromosome-bearing sperm cells with a staining media comprising a DNA selective dye;
contacting the stained sperm cell sample with a sheath fluid in a flow path;
altering a ratio of the viable X chromosome-bearing sperm cells to the viable Y chromosome-bearing sperm cells to form at least one altered sperm cell population; and
collecting the altered sperm cell population in a collection media comprising egg yolk at a wt./vol. concentration of 10% or less and a low-density lipoprotein additive, thereby reducing agglutination and improving health of the sperm cells in the collected sperm cell population.

2. The method of claim 1, wherein the step of altering a ratio of the viable X chromosome-bearing sperm cells to the viable Y chromosome-bearing sperm cells to form at least one altered sperm cell population comprises i) removing at least a portion of one of either the viable X chromosome-bearing sperm cells or the viable Y chromosome-bearing sperm cells from the sperm cell sample or ii) photo-damaging at least a portion of one of either the viable Y chromosome-bearing sperm cells or the viable X chromosome-bearing sperm cells.

3. The method of claim 1, further comprising the steps of injecting the stained sperm cell sample into a flow of sheath fluid;
exposing the stained sperm cell sample in the flow of sheath fluid to an electromagnetic radiation source that causes a detectable response in the DNA selective dye;
detecting the response of the DNA selective dye to the electromagnetic radiation exposure;
analyzing the detected response; and
classifying sperm cells in the sperm cell sample based on the analysis of the detected response.

4. The method of claim 1, wherein the egg yolk is at a wt./vol. concentration between 0.01% to 10%.

5. The method of claim 1, wherein the egg yolk is at wt./vol. concentration of between 0.01% to 5%.

6. The method of claim 1, wherein the egg yolk is at a wt./vol. concentration of between 0.01% to 1%.

7. The method of claim 1, wherein the egg yolk is at a wt./vol. concentration of between 0.01% to 0.5%.

8. The method of claim 1, wherein the egg yolk is at a wt./vol. concentration of between 0.01% to 0.05%.

9. The method of claim 1, wherein the egg yolk is at a wt./vol. concentration of 0.01%.

10. The method of claim 1, wherein the egg yolk is at a wt./vol. concentration of 0%.

11. The method of claim 1, wherein the low-density lipoprotein additive is extracted from pasteurized egg yolk.

12. The method of claim 1, wherein the low-density lipoprotein additive is at a wt./vol. concentration of between 0.5% to 30%.

13. The method of claim 1, wherein the low-density lipoprotein additive is at a wt./vol. concentration of between 0.5% to 10%.

14. The method of claim 1, wherein the low-density lipoprotein additive is at a wt./vol. concentration of between 0.5% to 5%.

15. The method of claim 1, wherein the low-density lipoprotein additive is at a wt./vol. concentration of between 1% to 5%.

16. The method of claim 1, wherein the low-density lipoprotein additive is at a wt./vol. concentration of 2.5%.

17. The method of claim 1, wherein the low-density lipoprotein additive is at a wt./vol. concentration of between 1% to 5% and the egg yolk is at a wt./vol. concentration of between 0.01% to 1%.

18. The method of claim 1, wherein the sperm cells are porcine sperm cells or bovine sperm cells.

19. The method of claim 1, wherein the step of collecting is conducted at a temperature less than 37° C.

20. The method of claim 1, wherein the step of collecting is conducted at a temperature less than 22° C.

21. The method of claim 1, wherein the low-density lipoprotein additive is extracted using polyethylene glycol.

22. The method of claim 1, wherein the collection media further comprises vitamin B or alpha-ketoglutarate.

23. A composition comprising:
egg yolk at a wt./vol. concentration of between 0.01% to 0.05%;
a low-density lipoprotein additive at a wt./vol. concentration of between 1% to 5%; and
a sperm cell sample comprising an altered ratio of viable X chromosome-bearing sperm cells to viable Y chromosome-bearing sperm cells.

24. The composition of claim 23, wherein the low-density lipoprotein additive is extracted from pasteurized egg yolk.

25. The composition of claim 23, wherein the sperm cell sample in the composition has an agglutination rank of 2 or less.
